(19) United States
(12) Patent Application Publication
(10) Pub. No.: US 2005/0106714 A1
(43) Pub. Date: May 19, 2005

(54) Rotatable reactor systems and methods

(76) Inventors: Andrey J. Zarur, Winchester, MA (US); Todd A. Basque, Danvers, MA (US); Derek T. Stevens, Boston, MA (US); Nicholas J. Flannery, Brockton, MA (US); Seth T. Rodgers, Somerville, MA (US); A. Peter Russo, Woburn, MA (US); Scott Miller, Somerville, MA (US); Ian K. MacGregor, Merrimack, NH (US)

Correspondence Address: Timothy J. Oyer, Ph.D.;Wolf, Greenfield & Sacks, P.C., 600 Atlantic Avenue, Boston, MA, 02210-2206, US

(21) Appl. No.: 10/927,789

(22) Filed: Aug. 27, 2004

Related U.S. Application Data
(63) Continuation-in-part of application No. 10/457,015, filed on Jun. 5, 2003.
(60) Provisional application No. 60/498,981, filed on Aug. 29, 2003.
(60) Provisional application No. 60/386,322, filed on Jun. 5, 2002.

(51) Int. Cl. C12M 3/00

(52) U.S. Cl. 435/288.4; 435/305.3

(57) ABSTRACT

The present invention generally relates to chips, particularly microfluidic chips, that are rotatable and/or have a generally circular or rotationally symmetric geometry. The chips may be substantially planar in certain instances. In some cases, the chips of the invention can have more than one reaction site, which can, for example, contain cells. The reaction site can be very small, in some cases with a volume of less than about 1 ml. Reactions, transport, and/or other manipulations within the chip can be facilitated by rotating the chip, for example, at tens, hundreds or thousands of revolutions per minute (RPM). In some cases, data may also be written to and/or read from the chip. The chips of the invention can be used, for example, to move fluid from one portion of a chip to another, to concentrate and/or separate a mixture (e.g., a cell suspension), to lyse or fractionate a cell, or the like.

### RELATED APPLICATIONS

This application claims the benefit U.S. Provisional Patent Application Ser. No. 60/498,981, filed Aug. 29, 2003, entitled “Rotatable Reactor Systems and Methods” by Zarur, et al.. This application also is a continuation-in-part of co-pending U.S. patent application Ser. No. 10/457,015, filed Jun. 5, 2003, entitled “Reactor Systems Having a Light-Interacting Component,” by Scott E. Miller, et al., which claims the benefit of co-pending U.S. Provisional Patent Application Ser. No. 60/386,322, filed Jun. 5, 2002, entitled “Reactor Having Light-Interacting Component,” by S. Miller, et al. All of these applications are incorporated herein by reference in their entirety.

### FIELD OF THE INVENTION

This invention generally relates to reactors and reactor systems, and in particular, to reactors and reactor systems that are rotatable and/or have a generally circular geometry.

### BACKGROUND OF THE INVENTION

A wide variety of reaction systems are known for the production of products of chemical and/or biochemical reactions. Chemical plants involving catalysis, biochemical fermenters, pharmaceutical production plants, and a host of other systems are well-known. Biochemical processing may involve the use of a live microorganism (e.g., cells) to produce a substance of interest.

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

Recently, as described in International Patent Application Serial No. PCT/US01/07679, published on Sep. 20, 2001 as WO 01/68257, entitled “Microreactors,” incorporated herein by reference, cells have also been cultured on a very small scale (i.e., on the order of a few milliliters or less), so that, among other things, many cultures can be performed in parallel.

### SUMMARY OF THE INVENTION

A variety of reactors and reactor systems that are rotatable and/or have a circular geometry are provided in the present invention, as well as reactors and reactor systems involving rotatable systems and/or having circular geometry (e.g., around an axis) to perform certain operations of interest. The subject matter of this invention involves, in some cases, interrelated products and/or uses, alternative solutions to a particular problem, and/or a plurality of different uses of a single system or article.

In one aspect, the invention includes an apparatus. The apparatus, in one set of embodiments, is defined, at least in part, by a substantially planar chip of generally circular shape (i.e., a “disc”) having a predetermined reaction site with a volume of less than about 1 ml, where the predetermined reaction site is constructed and arranged to maintain at least one living cell at the site. In another set of embodiments, the apparatus is defined, at least in part, by a disc or other rotatable chip having a predetermined reaction site, where the disc or chip comprises a medium capable of storing information. For example, the disc may have the general dimensions and/or structure of a compact disc (CD) or a digital versatile disc (DVD).

In another aspect, the invention is a method. In one set of embodiments, the method includes the steps of providing a disc or other rotatable chip comprising a predetermined reaction site having a volume of less than about 1 ml, where the predetermined reaction site is constructed and arranged to maintain at least one living cell at the site; and providing a material in the predetermined reaction site. The method, in another set of embodiments, includes a step of rotating a disc or other rotatable chip around an axis (which may be perpendicular to the plane of the disc or chip in some cases) to allow a material to flow to and/or from the reaction site, i.e., due to the action of centripetal forces acting on the material. The method, in yet another set of embodiments, includes a step of rotating a disc or other rotatable chip around an axis to concentrate a mixture. The method, in yet another set of embodiments, includes a step of rotating a disc or other rotatable chip around an axis to centrifuge cells located in the reaction site. The method, in yet another set of embodiments, includes a step of rotating a disc or other rotatable chip around an axis to break down (lyse) or disrupt the cells located in the reaction site. In some of these cases, the axis may intersect the disc or the chip near the center of the disc or chip, and/or be substantially perpendicular to the disc or chip.

In another aspect, the invention is directed to a method of making a chip and/or a reactor system, e.g., a rotatable disc or chip as described in any of the embodiments herein. In yet another aspect, the invention is directed to a method of using a chip and/or a reactor system, e.g., a rotatable disc or chip as described in any of the embodiments described herein.

Other advantages and novel features of the invention will become apparent from the following detailed description of various non-limiting embodiments of the invention when considered in conjunction with the accompanying drawings. In cases where the present specification and a document incorporated by reference include conflicting disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates one embodiment of the invention, showing a disc containing several reaction sites;

FIG. 2 illustrates a rotatable chip in accordance with another embodiment of the invention; and

FIG. 3 illustrates a non-circular rotatable chip, in accordance with yet another embodiment of the invention.

### DETAILED DESCRIPTION OF THE INVENTION

The present invention generally relates to chips, particularly microfluidic chips, that are rotatable and/or have a generally circular or rotationally symmetric geometry. The chips may be substantially planar in certain instances. In some cases, the chips of the invention can have more than one reaction site, which can, for example, contain cells. The reaction site can be very small, in some cases with a volume of less than about 1 ml. Reactions, transport, and/or other manipulations within the chip can be facilitated by rotating the chip, for example, at tens, hundreds or thousands of revolutions per minute (RPM). In some cases, data may also be written to and/or read from the chip. The chips of the invention can be used, for example, to move fluid from one portion of a chip to another, to concentrate and/or separate a mixture (e.g., a cell suspension), to lyse or fractionate a cell, or the like.

The following applications are incorporated herein by reference: International Patent Application No. PCT/US01/07679, filed Mar. 9, 2001, entitled “Microreactors,” by Zarur, et al.; U.S. Provisional Patent Application Ser. No. 60/282,741, filed Apr. 10, 2001, entitled “Microfermentor Device and Cell Based Screening Method,” by Zarur, et al.; U.S. patent application Ser. No. 10/119,917, filed Apr. 10, 2002, entitled “Microfermentor Device and Cell Based Screening Method,” by Zarur, et al.; International Patent Application No. PCT/US02/11422, filed Apr. 10, 2002, entitled “Microfermentor Device and Cell Based Screening Method,” by Zarur, et al.; U.S. Provisional Patent Application Ser. No. 60/386,323, filed Jun. 5, 2002, entitled “Materials and Reactors having Humidity and Gas Control,” by Rodgers, et al.; U.S. Provisional Patent Application Ser. No. 60/386,322, filed Jun. 5, 2002, entitled “Reactor Having Light-Interacting Component,” by Miller, et al.; U.S. patent application Ser. No. 10/223,562, filed Aug. 19, 2002, entitled “Fluidic Device and Cell-Based Screening Method,” by Schreyer, et al.; U.S. Provisional Patent Application Ser. No. 60/409,273, filed Sep. 24, 2002, entitled “Protein Production and Screening Methods,” by Zarur, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et al.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al.; an International Patent Application, filed Jun. 5, 2003, entitled “Materials and Reactor Systems having Humidity and Gas Control,” by Rodgers, et al.; U.S. patent application Ser. No. 10/457,015, filed Jun. 5, 2003, entitled “Reactor Systems Having a Light-Interacting Component,” by Miller, et al.; an International Patent Application, filed Jun. 5, 2003, entitled “Reactor Systems Having a Light-Interacting Component,” by Miller, et al.; U.S. patent application Ser. No. 10/457,017, filed Jun. 5, 2003, entitled “System and Method for Process Automation,” by Rodgers, et al.; U.S. patent application Ser. No. 10/456,929, filed Jun. 5, 2003, entitled “Apparatus and Method for Manipulating Substrates,” by Zarur, et al; an international patent application, filed Aug. 19, 2003, entitled “Determination and/or Control of Reactor Environmental Conditions,” by Miller, et al.; an international patent application, filed Aug. 19, 2003, entitled “Systems and Methods for Control of pH and Other Reactor Environmental Conditions,” by Miller, et al.; an international patent application, filed Aug. 19, 2003, entitled “Microreactor Architecture and Methods,” by Rodgers, et al.; and a commonly-owned U.S. Patent Application filed on even date herewith, entitled “Reactor with Memory Component.”

A “chip,” as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “membrane” is a three-dimensional material having any shape such that one of the dimensions is substantially smaller than the other dimensions. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or allows water vapor to permeate it, but at a permeability that is at least an order of magnitude less. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes are transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent. The membrane, in one embodiment, may be used to divide a reaction site constructed and arranged to support cell culture from a second portion, for example, a reservoir. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. Of course, those of ordinary skill in the art will be able to design other arrangements, having varying numbers of cell culture portions, reservoir portions, and the like, as further described below.

As used herein, a “substantially transparent” material (for example, a membrane) is a material that allows electromagnetic radiation to be transmitted through the material without significant scattering, such that the intensity of electromagnetic radiation transmitted through the material is sufficient to allow the radiation to interact with a substance on the other side of the material, such as a chemical, biochemical, or biological reaction, or a cell. In some cases, the material is substantially transparent to incident electromagnetic radiation ranging between the infrared and ultraviolet ranges (including visible light) and, in particular, between wavelengths of about 400-410 nm and about 1,000 nm. In some cases, the material may be transparent to electromagnetic radiation between wavelengths of about 400-410 nm and about 800 nm, and in some embodiments, the material may be substantially transparent to radiation between wavelengths of about 450 nm and 700 nm. The substantially transparent material may be able to transmit electromagnetic radiation in some cases such that a majority of the radiation incident on the material passes through the material unaltered, and in some embodiments, at least about 50%, in other embodiments at least about 75%, in other embodiments at least about 80%, in still other embodiments at least about 90%, in still other embodiments at least about 95%, in still other embodiments at least about 97%, and in still other embodiments at least about 99% of the incident radiation is able to pass through the material unaltered. In certain cases, the material is at least partially transparent to electromagnetic radiation within the above-mentioned wavelength range to the extent necessary to promote and/or monitor a physical, chemical, biochemical, and/or biological reaction occurring within a reaction site, for example as previously described. In other embodiments, the material may be transparent to electromagnetic radiation within the above-mentioned wavelength range to the extent necessary to monitor, observe, stimulate and/or control a cell within the reaction site.

As used herein, a “reactor” is the combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to promote and/or monitor a biological, chemical, or biochemical reaction, interaction, operation, or experiment at a reaction site, and which can be part of a chip. For example, a chip may include at least 5, at least 10, at least 20, at least 50, at least 100, at least 500, or at least 1,000 or more reactors.

Examples of reactors include chemical or biological reactors and cell culturing devices, as well as the reactors described in International Patent Application Serial No. PCT/US01/07679, published on Sep. 20, 2001 as WO 01/68257, incorporated herein by reference. Reactors can include one or more reaction sites or chambers. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the system in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, At least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a chip. The reaction site may be defined as a region where a reaction is allowed to occur; for example, the reactor may be constructed and arranged to cause a reaction within a channel, one or more chambers, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

In some cases, the reaction site containing cells may include a region containing a gas (e.g., a “gas head space”), for example, if the reaction site is not completely filled with a liquid. The gas head space, in some cases, may be partially separated from the reaction site, through use of a gas-permeable or semi-permeable membrane. In some cases, the gas head space may include various sensors for monitoring temperature, and/or other reaction conditions.

Many embodiments and arrangements of the invention are described with reference to a chip, or to a reactor, and those of ordinary skill in the art will recognize that the invention can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

In some embodiments, the reaction site may be defined by geometrical considerations. For example, the reaction site may be defined as a chamber in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site can be very small in certain embodiments. Specifically, the reaction site may have a volume of less than one liter, less than about 100 ml, less than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. The reaction site may have any convenient size and/or shape. In another set of embodiments, the reaction site may have a dimension that is 500 microns deep or less, 200 microns deep or less, or 100 microns deep or less.

In some cases, cells can be present at the reaction site. Sensor(s) associated with the chip or reactor, in certain cases, may be able to determine the number of cells, the density of cells, the status or health of the cell, the cell type, the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site, or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

Chips of the invention may also include a plurality of inlets and/or outlets that can receive and/or output any of a variety of reactants, products, and/or fluids, for example, directed towards one or more reactors and/or reaction sites. In some cases, the inlets and/or outlets may allow the aseptic transfer of compounds. At least a portion of the plurality of inlets and/or outlets may be in fluid communication with one or more reaction sites within the chip. In some cases, the inlets and/or outlets may also contain one or more sensors and/or actuators, as further described below. Essentially, the chip may have any number of inlets and/or outlets from one to tens of hundreds that can be in fluid communication with one or more reactors and/or reaction sites. Less than 5 or 10 inlets and/or outlets may be provided to the reactor and/or reaction site(s) for certain reactions, such as biological or biochemical reactions. In some cases each reactor may have around 25 inlets and/or outlets, in other cases around 50 inlets and/or outlets, in still other cases around 75 inlets and/or outlets, or around 100 or more inlets and/or outlets in still other cases.

As one example, the inlets and/or outlets of the chip, directed to one or more reactors and/or reaction sites may include inlets and/or outlets for a fluid such as a gas or a liquid, for example, for a waste stream, a reactant stream, a product stream, an inert stream, etc. In some cases, the chip may be constructed and arranged such that fluids entering or leaving reactors and/or reaction sites do not substantially disturb reactions that may be occurring therein. For example, fluids may enter and/or leave a reaction site without affecting the rate of reaction in a chemical, biochemical, and/or biological reaction occurring within the reaction site, or without disturbing and/or disrupting cells that may be present within the reaction site. Examples of inlet and/or outlet gases may include, but are not limited to, CO₂, CO, oxygen, hydrogen, NO, NO₂, water vapor, nitrogen, ammonia, acetic acid, etc. As another example, an inlet and/or outlet fluid may include liquids and/or other substances contained therein, for example, water, saline, cells, cell culture medium, blood or other bodily fluids, antibodies, pH buffers, solvents, hormones, carbohydrates, nutrients, growth factors, therapeutic agents (or suspected therapeutic agents), antifoaming agents (e.g., to prevent production of foam and bubbles), proteins, antibodies, and the like. The inlet and/or outlet fluid may also include a metabolite in some cases. A “metabolite,” as used herein, is any molecule that can be metabolized by a cell. For example, a metabolite may be or include an energy source such as a carbohydrate or a sugar, for example, glucose, fructose, galactose, starch, corn syrup, and the like. Other example metabolites include hormones, enzymes, proteins, signaling peptides, amino acids, etc.

The inlets and/or outlets may be formed within the chip by any suitable technique known to those of ordinary skill in the art, for example, by holes or apertures that are punched, drilled, molded, milled, etc. within the chip or within a portion of the chip, such as a substrate layer. In some cases, the inlets and/or outlets may be lined, for example, with an elastomeric material. In certain embodiments, the inlets and/or outlets may be constructed using self-sealing materials that may be re-usable in some cases. For example, an inlet and/or outlet may be constructed out of a material that allows the inlet and/or outlet to be liquid-tight (i.e., the inlet and/or outlet will not allow a liquid to pass therethrough without the application of an external driving force, but may admit the insertion of a needle or other mechanical device able to penetrate the material under certain conditions). In some cases, upon removal of the needle or other mechanical device, the material may be able to regain its liquid-tight properties (i.e., a “self-sealing” material). Non-limiting examples of self-sealing materials suitable for use with the invention include, for example, polymers such as polydimethylsiloxane (“PDMS”), natural rubber, HDPE, or silicone materials such as Formulations RTV 108, RTV 615, or RTV 118 (General Electric, New York, N.Y.).

In some embodiments, the chip of the present invention may include very small elements, for example, sub-millimeter or microfluidic elements. For example, in some embodiments, the chip may include at least one reaction site having a cross sectional dimension of no greater than, for example, 100 mm, 80 mm, 50 mm, or 10 mm. In some embodiments, the reaction site may have a maximum cross section no greater than, for example, 100 mm, 80 mm, 50 mm, or 10 mm. As used herein, the “cross section” refers to a distance measured between two opposed boundaries of the reaction site, and the “maximum cross section” refers to the largest distance between two opposed boundaries that may be measured. In other embodiments, a cross section or a maximum cross section of a reaction site may be less than 5 mm, less than 2 mm, less than 1 mm, less than 500 micrometers, less than 300 micrometers, less than 100 micrometers, less than 10 micrometers, or less than 1 micrometer or smaller. As used herein, a “microfluidic chip” is a chip comprising at least one fluidic element having a sub-millimeter cross section, i.e., having a cross section that is less than 1 mm. As one particular non-limiting example, a reaction site may have a generally rectangular shape, with a length of 80 mm, a width of 10 mm, and a depth of 5 mm.

While one reaction site may be able to hold and/or react a small volume of fluid as described herein, the technology associated with the invention also allows for scalability and parallelization. With regard to throughput, an array of many reactors and/or reaction sites within a chip, or within a plurality of chips, can be built in parallel to generate larger capacities. For example, a plurality of chips (e.g. at least about 10 chips, at least about 30 chips, at least about 50 chips, at least about 75 chips, at least about 100 chips, at least about 200 chips, at least about 300 chips, at least about 500 chips, at least about 750 chips, or at least about 1,000 chips or more) may be operated in parallel, for example, through the use of robotics, for example which can monitor or control the chips automatically. Additionally, an advantage may be obtained by maintaining production capacity at the small scale of reactions typically performed in the laboratory, with scale-up via parallelization. It is a feature of the invention that many reaction sites may be arranged in parallel within a reactor of a chip and/or within a plurality of chips. Specifically, at least five reaction sites can be constructed to operate in parallel, or in other cases at least about 7, about 10, about 30, about 50, about 100, about 200, about 500, about 1,000, about 5,000, about 10,000, about 50,000, or even about 100,000 or more reaction sites can be constructed to operate in parallel, for example, in a high-throughput system. In some cases, the number of reaction sites may be selected so as to produce a certain quantity of a species or product, or so as to be able to process a certain amount of reactant. Of course, the exact locations and arrangement of the reaction site(s) within the reactor or chip will be a function of the specific application.

Additionally, any embodiment described herein can be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters. Large volumes may be appropriate where the reactors and/or reaction sites are arranged in parallel within one or more chips, e.g., a plurality of reactors and/or reaction sites may be able to deliver a product to a collection chamber.

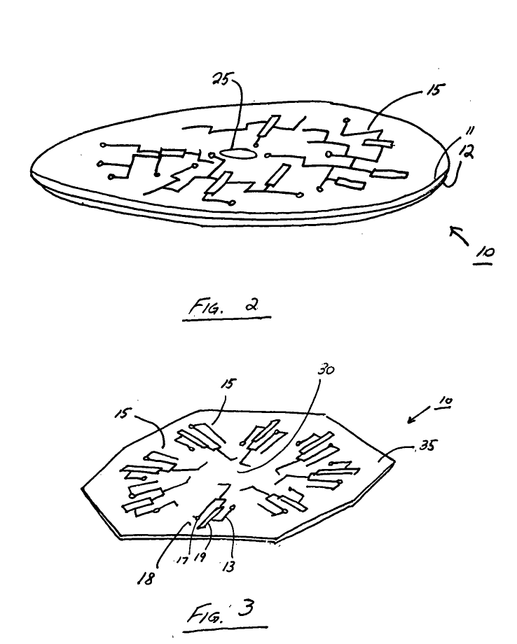

In some embodiments, the reaction site(s) and/or the channels in fluidic communication with the reaction site(s) are free of active mixing elements. In these embodiments, the reactor of the chip can be constructed in such a way as to cause turbulence in the fluids provided through the inlets and/or outlets, thereby mixing and/or delivering a mixture of the fluids, preferably without active mixing, where mixing is desired. Specifically, the reactor and/or reaction site(s) may include a plurality of obstructions in the flow path of the fluid that causes fluid flowing through the flow path to mix, for example, as shown in mixing unit 42 in FIG. 2. These obstructions can be of essentially any geometrical arrangement for example, a series of pillars. As used herein, “active mixing elements” is meant to define mixing elements such as blades, stirrers, or the like, which are movable relative to the reaction site(s) and/or channels themselves, that is, movable relative to portion(s) of the reactor defining a reaction site a or a channel.

Chips of the invention can be constructed and arranged such that they are able to be stacked in a predetermined, pre-aligned relationship with other, similar chips, such that the chips are all oriented in a predetermined way (e.g., all oriented in the same way) when stacked together. When a chip of the invention is designed to be stacked with other, similar chips, it often can be constructed and arranged such that at least a portion of the chip, such as a reaction site, is in fluidic communication with one or more of the other chips and/or reaction sites within other chips. This arrangement can find use in parallelization of chips, as discussed herein.

In one set of embodiments, the chip is constructed and arranged such that the chip is able to be stably connected to a microplate, for example, as defined in the 2002 SPS/ANSI proposed standard (e.g., a microplate having dimensions of roughly 127.76±0.50 mm by 85.48±0.50 mm). As used herein, “stably connected” refers to systems in which two components are connected such that a specific motion or force is necessary to disconnect the two components from each other, i.e., the two components cannot be dislodged by random vibration or displacement (e.g., accidentally lightly bumping a component). The components can be stably connected by way of pegs, screws, snap-fit components, matching sets of indentations and protrusions, or the like. A “microplate” is also sometimes referred to as a “microtiter” plate, a “microwell” plate, or other similar terms known to the art. The microplate may include any number of wells. For example, as is typically used commercially, the microplate may be a six-well microplate, a 24-well microplate, a 96-well microplate, a 384-well microplate, or a 1,536-well microplate. The wells may be of any suitable shape, for example, cylindrical or rectangular. The microplate may also have other numbers of wells and/or other well geometries or configurations, for instance, in certain specialized applications.

In another set of embodiments, one or more reaction sites may be positioned in association with a chip such that, when the chip is stably connected to other chips, one or more reaction sites of the chip are positioned or aligned to be in chemical, biological, or biochemical communication with, or chemically, biologically, or biochemically connectable with one or more reaction sites of the other chip(s) and/or one or more wells of the micropiate(s). “Alignment,” in this context, can mean complete alignment, such that the entire area of the side of a reaction site adjacent another reaction site or well completely overlaps the other reaction site or well, and vice versa, or at least a portion of the reaction site can overlap at least a portion of an adjacent reaction site or well. “Chemically, biologically, or biochemically connectable” means that the reaction site is in fluid communication with another reaction site or well (i.e., fluid is free to flow from one to the other); or is fluidly connectable to the other site or well (e.g., the two are separated from each other by a wall or other component that can be punctured or ruptured, or a valve in a conduit connecting the two can be opened); or the reaction site and other site or well are arranged such that at least some chemical, biological, or biochemical species can migrate from one to the other, e.g., across a semipermeable membrane. As examples, a chip may have six reaction sites that are constructed and arranged to be aligned with the six wells of a 6-well microplate when the chip is stably connected with the microplate (e.g., positioned on top of the microplate), a chip having 96 reaction sites may be constructed and arranged such that the 96 wells are constructed and arranged to be aligned with the 96 wells of a 96-well microplate when the chip is stably connected with the microplate, etc. Of course, in some cases, the chip may be constructed and arranged such that a single reaction site of the chip is aligned with more than one microplate well and/or more than one other reaction site, and/or such that more than one microplate well and/or more than one other reaction site is aligned with a single reaction site of the chip.

Chips of the invention also may be constructed and arranged such that at least one reaction site and/or reactor of the chip is in fluid communication with, and/or chemically, biologically, or biochemically connectable to an apparatus constructed and arranged to address at least one well of a microplate, for example, an apparatus that can add species to and/or remove species from wells of microplates, and/or can test species within wells of a microplate. In this arrangement, the apparatus may add and/or remove species to/from a reaction site of a chip, and/or test species at reaction sites. In this embodiment, the reaction sites typically are arranged in alignment with wells of the microplate.

Chips of the invention can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use. In this set of embodiments, the chip is constructed and arranged such that when the chip or reactor is filled with water and the inlets and/or outlets sealed, the chip or reactor has an evaporation rate of less than about 100 microliters per day, less than about 50 microliters per day, or less than about 20 microliters per day. In certain cases, a chip or reactor will exhibit an unmeasurable, non-zero amount of evaporation of water per day. The substantially liquid-tight chip or reactor can have a zero evaporation rate of water in other cases.

Chips of the invention can be fabricated using any suitable manufacturing technique for producing a chip having one or more reactors, each having one or multiple reaction sites, and the chip can be constructed out of any material or combination of materials able to support a fluidic network necessary to supply and define at least one reaction site. For example, the chip may be fabricated by etching silicon or other substrates, for example, via standard lithographic techniques. The chip may also be fabricated using microassembly or micromachining methods, for example, stereolithography, laser chemical three-dimensional writing methods, modular assembly methods, replica molding techniques, injection molding techniques, milling techniques, and the like as are known by those of ordinary skill in the art. The chip may also be fabricated by patterning multiple layers on a substrate, for example, as further described below, or by using various known rapid prototyping or masking techniques. Examples of materials that can be used to form chips include polymers, glasses, metals, ceramics, inorganic materials, and/or a combination of these. In some cases, the chip may be formed out of a material that can be etched to produce a reactor, reaction site and/or channel. For example, the chip may comprise an inorganic material such as a semiconductor, fused silica, quartz, or a metal. The semiconductor material may be, for example, but not limited to, silicon, silicon nitride, gallium arsenide, indium arsenide, gallium phosphide, indium phosphide, gallium nitride, indium nitride, other Group III/V compounds, Group II/VI compounds, Group III/V compounds, Group IV compounds, and the like, for example, compounds having three or more elements. The semiconductor material may also be formed out of combination of these and/or other semiconductor materials known in the art. In some cases, the semiconductor material may be etched, for example, via known processes such as lithography. In certain embodiments, the semiconductor material may have the from of a wafer, for example, as is commonly produced by the semiconductor industry.

In some embodiments, a chip of the invention may be formed from or include a polymer, such as, but not limited to, polyacrylate, polymethacrylate, polycarbonate, polystyrene, polyethylene, polypropylene, polyvinylchloride, polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The chip may also be formed from composite materials, for example, a composite of a polymer and a semiconductor material.

In one aspect of the invention, the chip may be secured in a holding apparatus. As used herein, “secure” means to affix an object to an apparatus such that the object will not be dislodged from the apparatus due to motion of the apparatus. For example, the holding apparatus may position, move, invert, rotate, revolve, agitate, stir, and/or vibrate the chip without dislodging it. The chip, of course, may be intentionally removed from the holding apparatus by an operator (e.g., a mechanical or automated device, or a human user). As one example, a chip or other substrate may be placed into a slot or a holder of a holding apparatus designed to secure the chip or other substrate during use of the apparatus. Optionally, mechanical restraints, such as hooks, guides, clips, fasteners, bands, or springs may be used to secure the chip to the apparatus. As another example, a chip may be secured to an apparatus via a clamp. As yet another example, a chip may be secured in an apparatus in such a way that the chip is able to move within the apparatus in some fashion without being dislodged from the apparatus due to motion of the apparatus.

In some embodiments, the chip, or at least a portion thereof, is rigid, such that the chip is sufficiently sturdy in order to be handled by commercially-available microplate-handling equipment, and/or such that the chip does not become deformed after routine use. Those of ordinary skill in the art are able to select materials or a combination of materials for chip construction that meet this specification, while meeting other specifications for use for which a particular chip is intended.

In certain embodiments, the chip may include a sterilizable material. For example, the chip may be sterilizable in some fashion to kill or otherwise deactivate biological cells (e.g., bacteria), viruses, etc. therein, before the chip is used or re-used. For instance, the chip may be sterilized with chemicals, radiated (for example, with ultraviolet light and/or ionizing radiation), heat-treated, or the like. Appropriate sterilization techniques and protocols are known to those of ordinary skill in the art. For example, in one embodiment, the chip is autoclavable, i.e., the chip is constructed and arranged out of materials able to withstand commonly-used autoclaving conditions (e.g., exposure to temperatures greater than about 100° C. or about 120° C., often at elevated pressures, such as pressures of at least one atmosphere), such that the chip, after sterilization, does not substantially deform or otherwise become unusable. Another example of a sterilization technique is exposure to ozone. In another embodiment, the chip is able to withstand ionizing radiation, for example, short wavelength, high-intensity radiation, such as gamma rays, electron-beams, or X-rays. In some cases, ionizing radiation may be produced from a nuclear reaction, e.g., from the decay of ⁶⁰Co or ¹³⁷CS.

In some embodiments of the invention, the chip, or a portion thereof, such as a data storage component, may be moisture-resistant, i.e., the chip or component can be exposed to water without adversely affecting the chip or component. For example, the chip or component could be exposed to a liquid comprising water, a humidified atmosphere (e.g., within an incubator), ice (e.g., within a freezer), or steam (e.g., within an autoclave), without substantial damage or deformation (i.e., such that the chip or component can no longer function for its intended use).

The chip, according to another set of embodiments, may be made of materials selected and arranged with respect to each other such that the chip is temperature-resistant. For instance, the chip, or a portion thereof, such as a data storage compartment, may be used at any temperature, for example, a temperature much colder or much warmer than room temperature (25° C.), such as a temperature of less than about 10° C., less than about 4° C., less than about 0° C., less than about −10° C., less than about −20° C., less than about −40° C., less than about −80° C., or less, or at a temperature of at least about 30° C., at least about 37° C., at least about 50° C., at least about 60° C., at least about 75° C., at least about 100° C., or at least about 150° C. or more. For example, the chip may be positioned within a freezer, a refrigerator, at room temperature, within an incubator or an autoclave, etc. Thus, as specific examples, data may be written to and/or read from a data storage compartment on a chip while the chip is within a freezer, a refrigerator, an incubator, etc.

In one set of embodiments, at least a portion of the chip may be fabricated without the use of adhesive materials. For example, at least two components of a chip (e.g., two layers of the chip if the chip is a multi-layered structure, a layer or substrate of the chip and a membrane, two membranes, an article of the chip and a component of a microfluidic system, etc.) may be fastened together without the use of an adhesive material. For example, the components may be connected by using methods such as heat sealing, sonic welding, via application of a pressure-sensitive material, and the like. In one embodiment, the components may be held in place mechanically. For example, screws, posts, cantilevers, etc. may be used to mechanically hold the chip (or a portion thereof) together. In other embodiments, the two components of the chip may be joined together using techniques such as, but not limited to, heat-sealing methods (e.g., or more components of the chip may be heated to a temperature greater than the glass transition temperature or the melting temperature of the component before being joined to other components), or sonic welding techniques (e.g., vibration energy such as sound energy may be applied to one or more components of the chip, allowing the components to at least partially liquefy or soften).

In another set of embodiments, two or more components of the chip may be joined using an adhesive material. As used herein, an “adhesive material” is given its ordinary meaning as used in the art, i.e., an auxiliary material able to fasten or join two other materials together. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid. For example, in one embodiment, the adhesive may be applied to the component(s) using transfer tape (e.g., a tape having adhesive material attached thereto, such that, when the tape is applied to the component, the adhesive, or at least a portion of the adhesive, remains attached to the component when the tape is removed from the component). In one set of embodiments, the adhesive maybe a pressure-sensitive adhesive, i.e., the material is not normally or substantially adhesive, but becomes adhesive and/or increases its adhesive strength under the influence of pressure, for example, a pressure greater than about 6 atm or about 13 atm (about 100 psi or about 200 psi). Examples of pressure-sensitive adhesives include AR Clad 7876 (available from Adhesives Research, Inc., Glen Rock, Pa.) and Trans-Sil Silicone PSA NT-1001 (available from Dielectric Polymers, Holyoke, Mass.)

In some embodiments of the invention, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. Of course, other components of the chip may be constructed without the use of adhesive materials, as previously discussed.

The term “determining,” as used herein, generally refers to the measurement and/or analysis of a substance (e.g., within a reaction site), for example, quantitatively or qualitatively, or the detection of the presence or absence of the substance. “Determining” may also refer to the measurement and/or analysis of an interaction between two or more substances, for example, quantitatively or qualitatively, or by detecting the presence or absence of the interaction. Examples of techniques suitable for use in the invention include, but are not limited to, gravimetric analysis, calorimetry, pressure or temperature measurement, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements, including nephelometry.

In one aspect of the invention, a differential force is applied to a chip or other reactor system, i.e., a force is applied to the chip such that different portions of the chip experience different magnitudes of the force thereon, and/or forces are applied to different portions of the chip in different directions. For example, a differential force(s) may be applied to a chip by rotating the chip about an axis that passes through the chip, which can promote centrifugal motion in a species associated with the chip, e.g., impart centripetal forces to the chip; a differential force(s) can be applied by applying differential electric or magnetic fields to the chip; or the like, which field(s) can act on the chip itself (e.g., if electrically and/or magnetically susceptible), and/or act on species on or within the chip, e.g. electrically-susceptible and/or magnetically-susceptible fluids).

A “differential” force or forces is one or a set of forces that involves simultaneous application of force to at least two different locations on the chip, and/or species within or on the chip at at least two different locations, in at least two different directions and/or magnitudes, where the force(s) acts directly on the different chip locations and/or different species in the at least two different directions and/or the at least two different magnitudes. This is to be distinguished from a force in one direction on a chip (e.g. applied by a pump to a fluid in a channel) which in turn indirectly imparts a force in a different direction to the same or different fluid at a different location in the channel (or a second, connected channel) oriented differently on the chip. Of course, this differential force can also act to impart a force in a direction different from one of the at least two initial directions by, e.g., a fluid in a channel oriented having a first orientation itself imparting a force to a fluid in the same channel or a connected, second channel having a first orientation. In one embodiment, a differential force is applied such that at at least a first location and a second location on the chip, the chip or any species in or on the chip at those two different locations will experience, simultaneously, a first force in a first direction and/or of a first magnitude and a second force in a second direction and/or of a second magnitude, respectively. A differential force can be determined by measuring the magnitude or absolute value and/or direction of a force on one portion of the chip, and comparing that force to the force applied to a different portion of the chip. A differential force or set of forces can be applied either internally (via action of a mechanism on or within the chip) or externally (via action of a mechanism external to the chip). For example, such a force can be applied externally by action of a mechanism that imparts movement to the chip, e.g. a rotational force to the chip through an axis intersecting the chip.

As discussed above, the present invention relates primarily to chips and other reactor systems that are rotatable. As used herein, an object that is “rotatable” is constructed and arranged to be turned about an axis passing through the object. For example, a rotatable chip may have a hole, a pivot, a gear, an indentation, etc., around which the chip is designed to be rotated around. If the chip is planar, the axis may be perpendicular to the plane of the chip, and in some cases, may go near or through the center of the chip, although it need not. Those of ordinary skill in the art will understand the meaning of a device constructed and arranged to be rotatable about an axis that passes through the object, which will include at least some component or aspect designed to facilitate such rotation, as distinguished from the simple possibility of rotating almost any object about an axis passing through it.

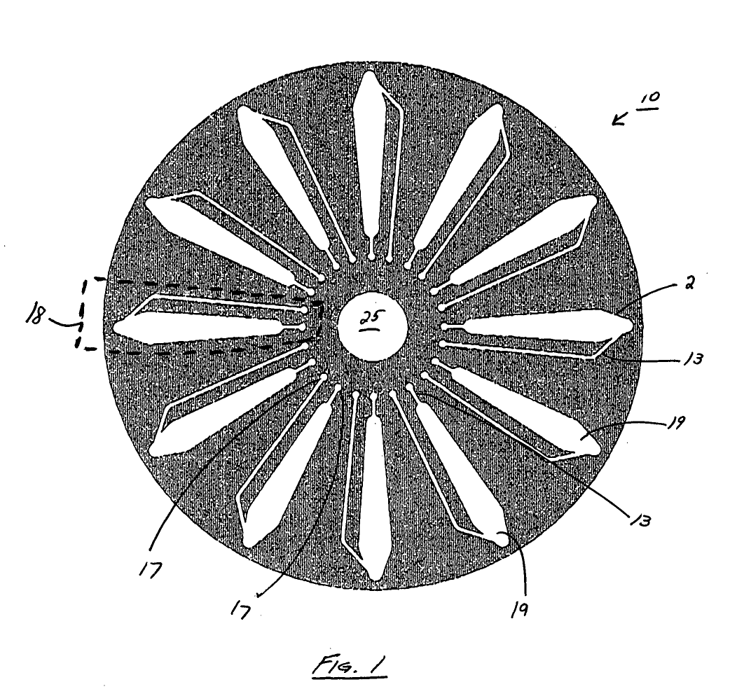

In some embodiments, the object may be rotationally symmetric, or at least generally rotationally symmetric, for example, as is shown in FIGS. 1 and 2. As used herein, an object that is “rotationally symmetric” means that the object can be rotated about an axis about which it is designed to rotate (e.g., a center point) by a number of degrees less than 360° such that, after the rotation, the object appears to be substantially the same as the object did before being rotated. For example, the object may be rotationally symmetric by 180° (two-fold symmetric), 120° (three-fold symmetric), 90° (four-fold symmetric), 72° (five-fold symmetric), 60° (six-fold symmetric), 45° (eight-fold symmetric), etc.

In one set of embodiments, the rotatable chip has a generally circular shape. In some cases, the rotatable chip may have the same general dimensions as a commercially available compact disc (CD) or digital versatile disc (DVD) (e.g., about 12 cm in diameter), a mini-CD (e.g., about 8 cm in diameter), etc.

The chip or other reactor system may be rotated at any suitable rotational speed to effect a desired manipulation within the chip, as further discussed below. For example, the chip may be spun at a rotational speed of about 5 revolutions per minute (“RPM”), about 10 RPM, about 30 RPM, about 100 RPM, about 300 RPM, about 800 RPM, about 1,000 RPM, about 1,500 RPM, about 2,000 RPM, about 5,000 RPM, or about 10,000 RPM or more in some cases. In some cases, the rotational speed may be variable, for example, the speed may be varied during an experiment or other manipulation performed on the chip.

By rotating the chip, reactions, transport (e.g., fluid transport), and/or other manipulations within the chip may be facilitated in some fashion. For example, a reaction may be initiated or terminated by rotating the chip (or changing the rotational speed of the chip), a material such as a fluid may be urged to flow from one portion of the chip to another and/or to or from the chip (for example, to and/or from a reaction site), a valve may be opened or closed, a fluid pathway may be activated or deactivated, a mixture may be concentrated and/or separated, a cell suspension may be separated, a cell may be lysed or fractionated, etc. For instance, the action of centripetal forces on a chip may cause a fluid to flow within the chip, which may be used to concentrate and/or dilute the fluid within the chip. As examples, the action of centripetal forces on a mixture (for example, two fluids, a fluid containing cells or other suspended particles, a mixture of solid particles, etc.) within the chip may cause the mixture to separate and/or form a gradient, for example, based on density differences between the components of the mixture. In another example, a fluid may be diluted, for example, by allowing centripetal force to cause the fluid to become diluted within a second fluid.

As another example, if a reactor includes cells, the chip may be rotated to manipulate the cells in some fashion. For example, the rotation of the chip may be used to separate or “spin down” the cells from a suspension. In other cases, the rotation of the chip may be used to disrupt or “lyse” the cells, and/or to separate or fractionate organelles and other intracellular components (e.g., at higher rotational speeds). Those of ordinary skill in the art will know of the appropriate rotational speeds, and the degree of centripetal force (g) that is required, to effect a desired manipulation of a cell.

As one particular example, as illustrated in FIG. 1, a substantially planar chip 10 (shown in top view) having a series of microfluidic elements thereon (which can, for example, include one or more reaction sites, channels, ports, valves, data storage components and the like) may be designed to be rotatable. In this example, chip 10 is designed to be rotated about a central hole 25 through which a spindle or the like can pass when the chip is connected to an apparatus designed to rotate it. The microfluidic elements on chip 10 can be grouped into a series of twelve reactors 18 (one reactor indicated within dotted lines). Each of the twelve reactors 18 in chip 10 has substantially the same structure and configuration (although not necessarily, but possibly, being identical); thus, rotation of chip 10 by 30° about central hole 25 does not alter the overall appearance of chip 10. In this figure, each reactor 18 includes a predetermined reaction site 19, a series of channels 13, and a series of ports 17. In this particular embodiment, each of the twelve reaction sites 19 takes the form of a substantially elongated compartment within chip 10, each extending radially outward between central hole 25 and the outer edge of chip 10. The reaction sites 19 are symmetrically arranged about central hole 25. Twenty-four ports 17 provide access to either of the two ends of reaction site 19, through a series of channels 13 that connect either end of reaction site 19 with ports 17. Ports 17, in this example, are arrayed about central hole 25, and are positioned such that each is the same distance from the center of chip 10. Of course, in other embodiments, ports 17 may be arranged in other configurations, for example, along the outer edge of chip 10, such that the ports are immediately next to the reaction sites, or the like. The chip may be fabricated, for example, such that it has the same general dimensions as a compact disc or a mini-compact disc, etc. A similar chip is illustrated in FIG. 2, having an upper layer 11 and a lower layer 12, within each of which various microfluidic elements can exist, and/or between which such elements can be defined. In this particular embodiment, microfluidic elements are not rotationally symmetric about central hole 25.

In FIG. 3, a non-circular rotationally-symmetric chip is shown, in accordance with another embodiment of the invention. In this embodiment, chip 10 has the shape of a regular octagon, and has a series of microfluidic elements 15 thereon that are eight-fold rotationally symmetrically distributed about a central pivot 30. Of course, in other embodiments, other relational symmetries are possible. The microfluidic elements 15 on chip 10 can be grouped into a series of eight reactors 18 (one reactor indicated within dotted lines). Each of the eight reactors 18 in chip 10 has substantially the same structure and configuration (although not necessarily being identical); thus, rotation of chip 10 by 45° about a pivot 30 does not alter the overall appearance of chip 10. Each reactor 18 includes a predetermined reaction site 19, a series of channels 13, and a series of ports 17. Of course, in other embodiments, other configurations of microfluidic elements within a chip or a reactor may be possible, depending on the specific application. For example, in some cases, each of the microfluidic elements may independently include one or more reaction sites, ports for fluidic access, etc. Chip 10 in the example shown in FIG. 3 also comprises a membrane 35, which may be, for instance, a substantially transparent membrane, a semipermeable membrane, a porous membrane, a humidity control membrane, etc. In this figure, membrane 25 is positioned on the surface of the chip to cover each of the microfluidic elements 15. Of course, in other embodiments, the membrane may only cover a portion of the chip.

In some embodiments of the invention, a chip may also include one or more data storage components, or in some cases, be defined by a data storage compartment. Any type of data may be included in the data storage compartment, and the data may be added to the chip at any time, for example, before, during, or after one or more experiments have been performed on the chip. In some cases, data may be pre-recorded in the data storage compartment before an experiment is performed on the chip. In other cases, data may be written to the chip during and/or after an experiment is performed on the chip. In some cases, the data storage compartment may include both pre-recorded data, and data that is added during or after use of the chip.

As used herein, a “memory component” or a “data storage component” is defined as an element that can be reliably uniquely associated with a chip (or predetermined set of chips), constructed and arranged to allow data to be stored to and/or retrieved from the element. A data storage component is associated with a chip, in accordance with one embodiment of the invention, if the data storage component is fastened to, embedded within, or integral with the chip, or otherwise will reliably travel with the chip as the chip is moved from place to place in the environment in which it is used, so that any data written to or retrieved from a particular data storage component will be reflective of some aspect or condition of the chip with which it is associated, and/or of one or more species with which the chip has been, is, or will be associated.

Various memory and/or data storage components are suitable and may include, but are not limited to, silicon integrated circuits, magnetic media, optical media, radio-frequency tags, smart cards, bar codes, and other kinds of data storage devices. In one embodiment, the data storage component includes a computer-readable medium, for example, a medium that stores information through electronic properties, magnetic properties, optical properties, etc. of the medium. Examples of computer-readable media include, but are not limited to, silicon and other semiconductor microchips or integrated circuits, bar codes, radio frequency tags or circuits, compact discs (e.g., in CD-R or CD-RW formats), digital versatile discs (e.g., in DVD+R, DVD−R, DVD+RW, or DVD−RW formats), insertable memory devices (e.g., memory cards, memory chips, memory sticks, memory plugs, etc.), “flash” memory, magnetic media (e.g., magnetic strips, magnetic tape, DATs, tape cartridges, etc.), floppy disks (e.g., 5.25 inch or 90 mm (3.5 inch) disks), optical disks, OCR readers, laser scanners, and the like. In one set of embodiments, the data storage component may be reversibly attached to and removed from the chip. In some embodiments, the data storage component may be volatile, i.e., some power is required by the data storage component to maintain the data therein. In other embodiments, however, the data storage component is non-volatile.

Data may be transmitted to/from the data storage component to the external data interface through any suitable method, for example, using any suitable data transfer protocol known to those of ordinary skill in the art. For example, data may be transferred by magnetic interactions, such as by using a magnetic medium; electrical interactions (i.e., through an electrical connection, for example, by inserting the chip and/or the data storage component into a socket on an external data interface); light interactions (including laser interactions), for example, an optical medium; radio frequency interaction, for example, a radio-frequency tag; etc. Examples of data transfer protocols include, but are not limited to, Bluetooth® data transfer protocols, 802.11a, b, or g, etc., ftp protocols, Internet protocols and the like.

In one embodiment, the memory or data storage component includes a data storage chip. As used herein, a “data storage chip” is a microchip or microprocessor to which data can be stored and/or retrieved. Typically, the data storage chip comprises a semiconductor and often contains electronic circuitry. Examples of typical semiconductors for data storage chips include, but are not limited to, silicon, germanium, Group III-V compounds (e.g., GaAs, InAs, GaP, InP, GaN, etc.), Group II/VI, Group III/V and Group IV semiconductors such as CdS, CdSe, InP, GaAs, and the like. Other semiconductor materials are described above. It should be understood that, as used herein, a “data storage chip” is to be distinguished from a “chip,” as defined above (i.e., an integral article that includes one or more reactors). In particular, in one set of embodiments, a chip of the invention can include one or more data storage chips.

More than one memory or data storage component may be present in association with a chip in some cases. For example, one or more memory or data storage components may be present within or on the chip, for instance, one or more memory or data storage components per reaction site. The data may be, for example, data related to a process or processes taking place within one or more of the reaction sites, identification data, parametric data (for example, data concerning when and where the chip was loaded, the contents of the chip, etc.) or the like. In some embodiments, the data may include physical, chemical, physicochemical, biological or biochemical data related to or associated with the reaction site. For example, the data may include experimental data, protocols and/or results, good manufacturing practices (“GMP”) data, data relevant to regulatory agencies, or the like.

The chip can also include a variety of components for sensing, actuation, or other activity. For example, the chip may include components such as a membrane, a lens, a light source, a waveguide, a circuit such as an integrated circuit, a reservoir (e.g., for a solution), a micromechanical or a MEMS (“microelectromechanical system”) component, a control system, or the like, for example, as further described below. In some embodiments, at least one, two, three or more components are integrally connected to the chip. In certain embodiments, all of the components are integrally connected to the chip.

As used herein, a “processor” or a “microprocessor” is any component or device able to receive a signal from one or more sensors, store the signal, and/or convert the signal into one or more responses for one or more actuators, for example, by using a mathematical formula or an electronic or computational circuit. The signal may be any suitable signal indicative of the environmental factor determined by the sensor, for example a pneumatic signal, an electronic signal, an optical signal, a mechanical signal, etc. The processor may be any device suitable for determining a response to the signal, for example, a mechanical device or an electronic device such as a semiconductor chip. The processor may be embedded and integrally connected with the reaction site or chip or separate from the reaction site or chip, depending on the application. In one embodiment, the processor is programmed with a process control algorithm, which can, for example, take an incoming signal from a sensor and convert the signal into a suitable output for an actuator. Any suitable algorithm(s) may be used within the processor, for example, a PID control system, a feedback or feedforward system, a fuzzy logic control system etc. The processor may be programmed or otherwise designed to control an environmental condition within the reaction site, for example, by manipulation of an actuator.

As used herein, an “actuator” is a device able to affect the environment within or proximate to one or more reaction sites, or in an inlet or outlet in fluid communication with one or more reaction sites. The actuator may be separate from, or integrally connected to the reaction site or chip. For example, in some embodiments, the actuator may include a valve or a pump able to control, alter, and/or prevent the flow of a substance or agent into or out of the reaction site, for example, a chemical solution, a buffering solution, a gas such as CO₂ or O₂, a nutrient solution, a saline solution, an acid, a base, a solution containing a carbon source, a nitrogen source, an inhibitor, a promoter, a hormone, a growth factor, an inducer, etc. The substance to be transported will depend on the specific application. In some cases, the pump may be external of the chip. As one example, the actuator may selectively open a valve that allows CO₂ or O₂ to enter the reaction site. In other cases, the pump may be internal of the chip. For example, the pump may be a piezoelectric pump or a mechanically-activated pump (e.g., activated by pressure, electrical stimulation, etc.). In one embodiment, the pump is activated by producing a gas within the chip, which may cause fluid flow within the chip; as examples, the gas may be produced by directing light such as laser light at a reactant to start a gas-producing reaction, or the gas may be produced by applying an electric current to a reactant (for instance, an electric current may be applied to water to produce gas). As another example, the actuator may include a pumping system that can create a fluid connection with a reaction site as necessary.

Other examples of components suitable for use with the invention include pylon-like obstructions placed in the flow path of a stream to enhance mixing within the chip, reactor and/or reaction site, or heating, separation, and/or dispersion units within the chip, reactor and/or reaction site. For example, if a heating unit is present, the heating unit may be a miniaturized, traditional heat exchanger.

In one set of embodiments, the present invention includes a membrane, such as a membrane that may be substantially transparent. If a membrane is present, it may be positioned anywhere in the reactor or chip. In one embodiment, the membrane is positioned such that it defines the surface of one or more reaction sites. In another embodiment, the membrane can be positioned such that it is in fluidic communication with one or more reaction sites of the reactor or chip. In some cases, the membrane may be positioned such that a pathway fluidly connecting a first reaction site with a second reaction site crosses the membrane. As used herein, a “substantially transparent” material (for example, a membrane) is a material that allows electromagnetic radiation to be transmitted through the material without significant scattering, such that the intensity of electromagnetic radiation transmitted through the material is sufficient to allow the radiation to interact with a substance on the other side of the material, such as a chemical, biochemical, or biological reaction, or a cell. In some cases, the material is substantially transparent to incident electromagnetic radiation ranging between the infrared and ultraviolet ranges (including visible light) and, in particular, between wavelengths of about 400-410 nm and about 1,000 nm. In some cases, the material may be transparent to electromagnetic radiation between wavelengths of about 400-410 nm and about 800 nm, and in some embodiments, the material may be substantially transparent to radiation between wavelengths of about 450 nm and 700 nm. The substantially transparent material may be able to transmit electromagnetic radiation in some cases such that a majority of the radiation incident on the material passes through the material unaltered, and in some embodiments, at least about 50%, in other embodiments at least about 75%, in other embodiments at least about 80%, in still other embodiments at least about 90%, in still other embodiments at least about 95%, in still other embodiments at least about 97%, and in still other embodiments at least about 99% of the incident radiation is able to pass through the material unaltered. In certain cases, the material is at least partially transparent to electromagnetic radiation within the above-mentioned wavelength range to the extent necessary to promote and/or monitor a physical, chemical, biochemical, and/or biological reaction occurring within a reaction site, for example as previously described. In other embodiments, the material may be transparent to electromagnetic radiation within the above-mentioned wavelength range to the extent necessary to monitor, observe, stimulate and/or control a cell within the reaction site.

In yet another set of embodiments, the membrane may be a porous membrane having, for example, a number-average pore size of greater than about 0.03 micrometers and less than about 2 micrometers. In other embodiments, the pore size of the membrane may be less than about 1.5 micrometers, less than about 1.0 micrometers, less than about 0.75 micrometers, less than about 0.5 micrometers, less than about 0.3 micrometers, less than about 0.1 micrometers, less than about 0.07 micrometers, and in other embodiments, less than about 0.05 micrometers. In certain cases, the pores are also greater than 0.03 micrometers or greater than 0.08 micrometers.

In certain embodiments, the membrane may be formed out of a substance that has a number-average pore size, as previously described, that is also substantially transparent, as previously described. For example, the porous substantially transparent membrane may include polymers such as polyethylene terephthalate (PET), polysulfone, polycarbonate, acrylics such as polymethyl methacrylate, polyethylene, polypropylene, and the like. In one embodiment, the substantially transparent membrane is a polyethylene terephthalate membrane having a pore size of 2 micrometers or less, for example, a ROTRAC® capillary membrane made by Oxyphen U.S.A., Inc. (New York, N.Y.).

In some cases, the membrane includes a humidity control material, for example, having a permeability and/or a permeance to one or more gases that corresponds to a range acceptable for culturing certain cells. For example, the humidity control material may have a permeability and/or permeance to oxygen high enough, and/or a permeability and/or permeance to water vapor low enough, to allow cell culturing. Those of skill in the art will be able to identify specific ranges of permeabilities of certain materials appropriate for successfully culturing particular cells and cell lines, as well as larger cellular groups, such as microbial and mammalian cells, tissues, tissue engineering constructs, etc.

For example, in one embodiment, the control system comprises a membrane including a humidity control material, for example, in a membrane having a permeability to oxygen high enough, and a permeability to water vapor low enough, to allow cell culturing. In one set of embodiments, the humidity control material may include a polymer (e.g., a single polymer type, a co-polymer, a polymer blend, a polymer derivative, etc.). Examples of polymers that may be used within the humidity control material include, but are not limited to, polyfluoroorganic materials such as polytetrafluoroethylenes (e.g., such as those marketed under the name TEFLON® by DuPont of Wilmington, Del., for example, TEFLON® AF) or certain amorphous fluoropolymers; polystyrenes; PP; silicones such as polydimethylsiloxanes; polysulfones; polycarbonates; acrylics such as polymethyl acrylate and polymethyl methacrylate; polyethylenes such as high-density polyethylenes (“HDPE”), low-density polyethylenes (“LDPE”), linear low-density polyethylenes (“LLDPE”), ultra low-density polyethylenes (“ULDPE”) etc.; PET; polyvinylchloride (“PVC”) materials, such as those marketed under the name SARAN® by Dow Chemical Co. of Midland, Mich.; nylons such as that marketed under the name DARTEK® by Dupont; a thermoplastic elastomer, and the like. Another example of a suitable material is a BIOFOIL® polymer membrane, made by VivaScience (Hannover, Germany). In one embodiment, the polymer may be poly(4-methylpentene-1) (“PMP”),which, in some cases, may have a permeability coefficient for oxygen of about 317.2 (m³STP m/s m Pa). Examples of PMPs include those marketed under the name TPX™ by Mitsui Plastics (White Plains, N.Y.). In other embodiments, the polymer may be poly(4-methylhexene-1), poly(4-methylheptene-1) poly(4-methyloctene-1), etc. In another embodiment, the polymer may be poly(1-trimethylsilyl-1-propyne) (“PTMSP”), which, in some cases, may have a permeability coefficient for oxygen of about 5.78×10⁵ (cm³_{STP} mm/m² day atm). In some cases, copolymer of these and/or other polymers may be used in the humidity control material.

Examples of permeability ranges of a humidity control material for use in the invention, for example for use in culturing a broad range of cells, include a permeability to oxygen greater than about 100 (cm³_{STP} mm/m² atm day), and a permeability to water vapor less than about 6×10⁻⁶ (cm³_{STP} mm/m² atm day). As used herein, “STP” refers to “standard temperature and pressure,” referring to a temperature of 273.15K (0° C.) and a pressure of about 10⁵ Pa (1 atm). In another embodiment, the humidity control material may have a permeability to water that is less than about 100 (cm³_{STP} mm/m² atm day) and, in other embodiments, less than about 30 (cm³_{STP} mm/m² atm day) or less than about 10 (cm³_{STP} mm/m² atm day), and an oxygen permeability of at least about 6×10⁶ (cm³_{STP} mm/m² atm day), and in some embodiments, at least about 1×10⁷ (cm³_{STP} mm/m² atm day), and in other embodiments greater than about 3×10⁷ (cm³_{STP} mm/m² atm day) or 1×10⁸ (cm³_{STP} mm/m² atm day). Any combination of oxygen permeability and water vapor permeability listed herein can be used. For microbial cells, an example of a suitable range of oxygen permeability is provided by a membrane having a permeability to oxygen permeability greater than about 1×10³ (cm³_{STP} mm/m² atm day) and/or a permeability to water vapor is less than about 6×10⁶ (cm³_{STP} mm/m² atm day). For mammalian cells, an example suitable range is provided by a membrane of the invention having a permeability to oxygen greater than about 100 (cm³_{STP} mm/m² atm day) and a permeability to water vapor lower than about 1×10⁵ (cm³_{STP} mm/m² atm day). In one embodiment, the present invention achieves a certain permeability by combining two or more layers or portions of material. For example, in one embodiment where the humidity control material comprises at least two layers, the layers may be formed out of the same or distinct polymers. In some embodiments, the area and thickness of the humidity control membrane, or a layer thereof, may be used to select a desired level of permeability. The humidity control material may also be substantially translucent or transparent in some cases.

In one set of embodiments, a chip of the invention may include a structure adapted to facilitate the reactions or interactions that are intended to take place therein (e.g., within a reaction site). For example, where a chip is intended to function as one or more bioreactors for cell culturing, the chip may include structure(s) able to improve or promote cell growth. For instance, in some cases, a surface of a reaction site may be a surface able to promote cell binding or adhesion, or the reactor and/or reaction site within the chip may include a structure that includes a cell adhesion layer, which may include any of a wide variety of hydrophilic, cytophilic, and/or biophilic materials. As examples, the surface may be ionized, or coated and/or micropatterned with any of a wide variety of hydrophilic, cytophilic, and/or biophilic materials, for example, materials having exposed carboxylic acid, alcohol, and/or amino groups. Examples of materials that may be suitable for a cell adhesion layer include, but are not limited to, polyfluoroorganic materials, polyester, PDMS, polycarbonate, polystyrene, and aluminum oxide. As another example, the structure may include a layer coated with a material that promotes cell adhesion, for example, an RGD peptide sequence, or the structure may be treated in such a way that it is able to promote cell adhesion, for example, the surface may be treated such that the surface becomes relatively more hydrophilic, cytophilic, and/or biophilic. In some embodiments, it may be desired to modify the surface of a cell adhesion layer, for instance with materials that promote cell adhesion, for example, by attachment, binding, soaking or other treatments. Example materials that promote cell adhesion include, but are not limited to, fibronectin, laminin, albumin or collagen. In other embodiments, for example, where certain types of bacteria or anchorage-independent cells are used, the surface may be formed out of a hydrophobic, cytophobic, and/or biophobic material, or the surface may be treated in some fashion to make it more hydrophobic, cytophobic, and/or biophobic, for example, by using aliphatic hydrocarbons and/or fluorocarbons.

In some embodiments, the chip may include a “light-interacting component,” i.e., a component that interacts with light, for example, by producing light, reacting to light, causing a change in a property of light, directing light, altering light, etc. As used herein, a “light-interacting component” is a component that interacts with light in some fashion related to chip and/or reactor function, for example, by producing light, reacting to light, causing a change in a property of light, directing light, altering light, etc., in a manner that affects a sample within a chip or reactor and/or determines something about the sample (the sample's presence, a characteristic of the sample, etc.). In one embodiment, the component produces light, such as in a light-emitting diode (“LED”) or a laser. In another embodiment, the light-interacting component is a component that is sensitive to light or responds to light, such as a photodetector or a photovoltaic cell. In yet another embodiment, the light-interacting component manipulates or alters light in some fashion, for example, by focusing or collimating light, or causing light to diverge, such as in a lens, or spectrally dispersing light, such as in a diffraction grating or a prism. In another embodiment, the light-interacting component transmits or redirects the direction of light in some fashion, such as along a bent path or around a corner, for example, as in a waveguide or mirror. In yet another embodiment, the light-interacting component alters a property of light incident on the component, such as the degree of polarization or the frequency, for example, as in a polarizer or an interferometer. Other devices, or combinations of devices, are also possible. In general, the term “light-interacting component” does not encompass components or devices that passively transmit light without significant modification, alteration, or redirection, such as air, or a plane of glass or plastic (e.g., a “window”). The term “light-interacting component” also does not generally encompass components that passively absorb essentially all incident light without a response, such as would be found in an opaque material.

The light-interacting component may include a waveguide in some cases. The term “waveguide” is given its ordinary meaning in the art and may include optical fibers. A waveguide is generally able to receive light and guide or transmit a portion of that light to a destination not within “line-of-sight” communication (although a waveguide can transmit light to a line-of-sight region), e.g., around bends, corners, and similar obstacles without substantial losses.

In some embodiments, a waveguide may include a “core” region of material embedded or surrounded, at least in part, by a second “cladding” material, which may have a lower refractive index than the core region. The core may have any shape, for example, a slab, a strip, or a cylinder of material.

The waveguide, or at least a portion of the waveguide, may be fashioned out of any material able to transmit or light to or from the reaction site. The waveguide may be substantially transparent, or translucent in some cases. In some embodiments, the waveguide may be formed out of a silicon-based material, for example, glass, ion-implanted glass, quartz, silicon, silicon oxide, silicon nitride, silicon carbide, polysilicon, coated glass, conductive glass, indium-tin-oxide glass and the like. In other embodiments, the waveguide may comprise other transparent or translucent organic or inorganic materials. For example, in certain embodiments, the waveguide may comprise a polymer including, but not limited to, polyacrylate, polymethacrylate, polycarbonate, polystyrene, polypropylene, polyethylene, polyimide, polyvinylidene fluoride, an ion-exchanged polymer, and fluorinated derivatives of the above. Combinations, blends, or copolymers are also possible.

In one embodiment, the waveguide or a portion thereof may be surrounded by or coated with a highly reflective material, for example, silver or aluminum. In another embodiment, the waveguide may be fashioned such that it comprises a central material (e.g., a core) having a first index of refraction, and a surrounding material (e.g., a cladding) having a second index of refraction. The cladding may have an index of refraction that is less than the index of refraction of the central material. In yet another embodiment, the index of refraction of the core or the cladding may vary over the cross section. As one example, the core may be a graded index optical fiber, where the index of refraction is generally highest near the center of the core.

As one example of a waveguide, both the central and surrounding materials forming the waveguide may each be a glass. As another example, a waveguide may be formed out of a polymer and a silicon-based material, such that the material with the lower index of refraction surrounds the material with the higher index of refraction. As yet another example, the waveguide may be constructed out of a single material surrounded by, for example, air or a portion of the chip having a higher index of refraction than the waveguide, thus resulting in a condition where total internal reflection may occur at the waveguide/air or waveguide/chip interface.

The waveguide may be constructed by any suitable technique known to those of ordinary skill in the art, for example, by milling, grinding, or machining (e.g., by cutting or etching a channel into a chip substrate, then depositing material into the channel, optionally using a sealant). The waveguide may also be formed, for example, by depositing layers of materials during the chip fabrication process. The deposited material, in some cases, can have a higher index of refraction than the surrounding reactor substrate, thus forming a general core-cladding structure, as previously described. The waveguide may also be constructed by laser etching of materials forming the chip, such as glass or plastic, in such a way as to manipulate/alter the refractive index, relative to the surrounding material. In some cases, the refractive index of the etched/non-etched portion may be controlled so as to create a core-cladding structure.

In some embodiments, the light-interacting component may be, or include, a source of light. The light source may be any light source in optical communication with the reaction site. For example, the light source may be external or ambient light, a coherent or monochromatic beam of light such as created in an LED, or a laser such as a semiconductor laser or a quantum well laser. The light source may be integrally connected with a portion of the chip, for example, in a laser diode fabricated as part of the chip, or the light source may be separate from the chip and not integrally connected with it, but still positioned so as to allow optical communication with the reaction site. The light source may produce a single wavelength or a substantially monochromatic wavelength, or a wide range of wavelengths, as previously described. The source of light, in certain embodiments, may also be generated in a chemical reaction or a biological process, such as a chemical reaction that produces photons, for example, a reaction involving GFP (“green fluorescence protein”) or luciferase, or through fluorescence or phosphorescence. For example, incident electrons, electrical current, friction, heat, chemical or biological reactions may be applied to generate light, for example, within a sample located within a reaction site, or from a reaction center located within the chip in optical communication with the reaction site.

In certain cases, the light-interacting component may include a filter, for example, a low pass filter, a high pass filter, a notch filter, a spatial filter, a wavelength-selecting filter, or the like. The filter may be able to, for example, substantially reduce or eliminate a portion of the incident light. For example, the filter may eliminate or substantially reduce light having a wavelength below about 350 nm or greater than about 1000 nm. In another embodiment, the filter may be able to reduce noise within the incident light, or increase the signal-to-noise ratio of the incident light. In still another embodiment, the filter may be able to polarize the incident light, for example, linearly or circularly.

In some embodiments, the light-interacting component may include an optical element in optical communication with the reaction site. As used herein, an “optical element” refers to any element or device able to alter the pathway of light entering or exiting the optical element, for example, by focusing or collimating the light, or causing the light to diverge. For example, the optical element may focus the incident light to a single point or a small region, or the optical element may collimate or redirect divergent beams of light to form a parallel or converging beams of light. The term “focus” generally refers to the ability to cause rays of light to converge to a point or a small region. The term “collimate” generally refers to the ability to increase the convergence of rays of light, not necessarily to a point or a small region, for example, such that the beam focuses at an infinite distance. As one example, diverging beams of light may be collimated into parallel beams of light. In certain embodiments, the optical element may disperse or cause light to diverge, for example, as in a diverging lens. In other embodiments, the optical element may be, for example, a beamsplitter, an optical coating (e.g., a dichroic, an antireflective, or a reflective coating), an optical grating, a diffraction grating, or the like.

In one set of embodiments, the optical element may be a lens. The lens may be any lens, such as a converging or a diverging lens. The lens may be, for example, a meniscus, a piano-convex lens, a plano-concave lens, a double convex lens, a double concave lens, a Fresnel lens, a spherical lens, an aspheric lens, a binary lens, or the like. The optical element may also be a mirror, such as a planar mirror, a curved mirror, a parabolic mirror, or the like. In other embodiments, the optical element may cause light to disperse, for example, as in a diffraction grating or a prism.

In certain cases, a material having a different index of refraction may be used. For example, in embodiments in which light reaches the optical element through a waveguide, the optical element may be a material having a different index of refraction than the waveguide. In some cases, the index of refraction of the optical element will be about the same as or more than the index of refraction of the waveguide.

In some cases, a material having a graded index of refraction (a “GRIN” material) may be used as an optical element. The GRIN material may minimize the amount of divergence inherent in light reaching the GRIN material. For example, a material of uniform thickness can be made to act as a lens by varying its refractive index along a cross section of the element. In one embodiment, the GRIN material may redirect divergent rays of light into a parallel arrangement. In another embodiment, the GRIN material does not necessarily have a uniform thickness, and a combination of the graded index of refraction of the material and the shape of the material may be used to focus or collimate the light.

The light-interacting component, in some embodiments, may include a component that is able to convert light to electricity, such as a photosensor or photodetector, a photomultiplier, a photocell, a photodiode such as an avalanche photodiode, a photodiode array, a CCD chip (“charge-coupled device”) or the like. The component may be used, in some cases, to determine the state or condition of a substance within a reaction site, for example, through emission (including fluorescence or phosphorescence), absorbance, scattering, optical density, polarization measurements, or other measurements, including using the human eye.

In some embodiments of the invention, a reactor and/or a reaction site within a chip may be constructed and arranged to maintain an environment that promotes the growth of living cells. In embodiments where one or more cells are used in the reaction site, the cells may be any cell or cell type. For example, the cell may be a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used in the reaction site. The precise environmental conditions necessary in the reaction site for a specific cell type or types may be determined by those of ordinary skill in the art.

In some cases, the reactor and/or a reaction site may be constructed and arranged to promote the sustained growth of living cells within the reactor or reaction site. For example, the reactor and/or reaction site may sustain the growth of living cells therein for at least about 1 day, and in some cases, at least about 2 days, at least about 3 days, at least about 4 days, at least about 5 days, at least about a week, at least about 10 days, at least about 2 weeks, at least about 4 weeks, or longer in certain cases.

In some instances, the cells may produce chemical or biological compounds of therapeutic and/or diagnostic interest. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest.

In certain cases, a reactor and/or a reaction site within a chip may be constructed and arranged to prevent, facilitate, and/or determine a chemical or a biochemical reaction with the living cells within the reaction site (for example, to determine the effect, if any, of an agent such as a drug, a hormone, a vitamin, an antibiotic, an enzyme, an antibody, a protein, a carbohydrate, etc. on a living cell). For example, one or more agents suspected of being able to interact with a cell may be added to a reactor and/or a reaction site containing the cell, and the response of the cell to the agent(s) may be determined, using the systems and methods of the invention.

In some cases, the cells may be sensitive to light. For example, the cell may be a plant cell that responds to a light stimulus or is photosynthetic. In another embodiment, the light may be used to grow cells, such as mammalian cells sensitive to light, or plant cells. In yet another embodiment, the cell is a bacterium that is attracted to or is repelled by light. In another embodiment, the cell is an animal cell having a light receptor or other light-signaling response, for example, a rod cell or a cone cell. In yet another embodiment, the cell is a genetically engineered cell having a light receptor or another light-sensitive molecule, for example, one that decomposes or forms reactive entities upon exposure to light, or stimulates a biological process to occur. In other cases, the cell may be insensitive to light; light applied to the chip may be used for analysis of the cells, for example, detection, imaging, counting, morphological analysis, or spectroscopic analysis. In still other cases, the light may be used to kill the cells, for example, directly, or by inducing an apoptotic reaction.

In some embodiments, the chip is constructed and arranged such that cells within the chip can be maintained in a metabolically active state, for example, such that the cells are able to grow and divide. For instance, the chip may be constructed such that one or more additional surfaces can be added to the reaction site, for example, as in a series of plates, or the chip may be constructed such that the cells are able to divide while remaining attached to a substrate. In some cases, the chip may be constructed such that cells may be harvested or removed from the chip, for example, through an outlet of the chip, or by removal of a surface from the reaction site, optionally without substantially disturbing other cells present within the chip. The chip may be able to maintain the cells in a metabolically active state for any suitable length of time, for example, 1 day, 1 week, 30 days, 60 days, 90 days, 1 year, or indefinitely in some cases.

In one set of embodiments, the chip is able to control an environmental factor associated with a reaction site by transporting an agent into or proximate the reaction site. Control of the delivery of the agent to the reaction site, in certain instances, may be used to control the environmental factor. In some cases, the chip is able to control the environmental factor without directly contacting the reaction site to an external or unsterilized agent, such as a liquid. As used herein, an “environmental factor” or an “environmental condition” is a detectable and/or measurable condition (e.g., by a sensor) of the environment within and/or associated with a reaction site, such as the temperature or pressure. The factor or condition may be detected and/or measured within the reaction site, and/or at a location proximate to the reaction site (e.g., upstream or downstream of the reaction site) such that the environmental condition within the reaction site is known and/or controlled. For example, the environmental factor may be the concentration of a gas or a dissolved gas within the reaction site or associated with the reaction site (for example, upstream or downstream of the reaction site, separated from the reaction site by a membrane, etc.). The gas may be, for example, oxygen, nitrogen, water (i.e., the relative humidity), CO₂, or the like. The environmental factor may also be a concentration of a substance in some cases. For example, the environmental factor may be an aggregate quantity, such as molarity, osmolarity, salinity, total ion concentration, pH, or color. The concentration may also be the concentration of one or more compounds present within the reaction site, for example, an ion concentration such as sodium, potassium, calcium, iron or chloride ions; or a concentration of a biologically active compound, such as a protein, a lipid, or a carbohydrate source (e.g., a sugar) such as glucose, glutamine, pyruvate, apatite, an amino acid or an oligopeptide, a vitamin, a hormone, an enzyme, a protein, a growth factor, a serum, or the like. In some embodiments, the substance within the reaction site may include one or more metabolic indicators, for example, as would be found in media, or as produced as a waste products from cells.

Thus, in some cases, the environmental factor within the reaction site may be altered and/or controlled without directly contacting the reaction site with a liquid agent. In one embodiment, the chip may be constructed to allow an agent to permeate or diffuse into the reaction site. For instance, the reaction site may include a component such as a wall or a layer of the chip, through which an agent is able to permeate. The agent may be able to alter and/or control one or more environmental factors. For instance, the agent may be a non-pH-neutral composition or a pH-altering agent. As another example, the component may include a membrane, such as an osmotic membrane or a semipermeable membrane (e.g., with respect to the agent) that the agent is able to permeate across. In some cases, the component may be chemically or physically inert with respect to the agent. In certain instances, a flow of agent may occur on one side of the component. In some embodiments, the flow of agent on one side of the component may occur along a meandering or non-straight pathway, for example, to increase the time of contact between the agent and the component.

In some embodiments where a component of the chip (e.g., a layer or a membrane) comprises a polymer that a molecule (e.g. a small molecule) is able to permeate, the polymer may be or include, for example, nylon, polyethylene, polypropylene, polycarbonate, polydimethylsiloxane, or copolymers or blends. In another set of embodiments, the component may include a polymer substantially impermeable to the agent being transported, but the component may be constructed or designed to allow transport of the agent to occur, for example, through a region that is porous or contains a number of channels. In yet other embodiments, the component may be impermeable to the agent being transported, but the component may be converted to a permeable form upon the addition of a permeabilizing agent. As used herein, “permeation” and “permeate” refer to any suitable non-bulk transport process. A non-bulk transport, with respect to a substrate, generally is a transport process where substantial convection or bulk flow does not occur within the substrate. For example, permeation of the agent may occur through passive diffusion, for example, through the bulk material of a component or through pores or other interstices that may exist within the component; or the transport may be facilitated or enhanced in some manner, for instance, through osmosis, electrodiffusion, electroosmosis, percolation, or through the use of a permeation-enhancing compound within the component. In some embodiments, transport of the agent may be facilitated using an externally-applied field, such as an electrical, magnetic, or a centripetal field.

In some embodiments, the component may be designed to transport an agent therethrough within a given period of time or under a certain condition. In these cases, the exact thickness, density, porosity, tortuosity, composition, or other characteristics of the component may be determinable by those of ordinary skill in the art. For example, in some cases, the diffusion of the agent across the component may be generally Fickian, and the time it takes the agent to diffuse across the component may be determined using Fick's Law. In certain cases, transport of the agent across the component may be relatively rapid, for example, in cases where a relatively thin component is used. For instance, the component may be constructed such that an agent is transported therethrough in less than about 10 minutes, less than about 5 minutes, less than about 3 minutes, or less than about 1 minute, depending on the application.

In some embodiments, the environmental factor within the reaction site may be altered by generating one or more agents within the chip, for example, from one or more precursors. The agent(s) may interact with, or alter in some way, an environmental factor within the reaction site. In one embodiment, the agent may be generated within the reaction site. In another embodiment, the agent may be generated elsewhere within the chip and transported to the reaction site in some fashion, for instance, fluidically. For example, the chemical agent may be produced and/or stored within a different compartment associated with or external of the chip (e.g., as in a reservoir), then transported to the reaction site, for instance, through a channel or other fluidic connection, or by allowing it to permeate or diffuse across a membrane or another component. In one embodiment, the agent may be generated in a location proximate the reaction site, e.g., the agent may be generated in a location where it can be readily transferred or transported to the reaction site, for example, within a few seconds or tens of seconds. In another embodiment, the agent may be a gas that is transported to the reaction site, for example, through a membrane, or over a barrier that prevents liquid communication between the compartment and the reaction site, while non-gaseous products may be prevented from entering the reaction site. In certain embodiments of the invention, the reaction of the precursor(s) that produces the agent may be externally initiated. For example, a light source, such as a laser, may be applied to the precursor(s), or other energy sources such as electrical current or heat may be used to initiate a reaction of the precursor(s). In yet another embodiment, a fluidic connection may be created between the compartment and the reaction site, for example, reversibly. For instance, the fluidic connection may be created by opening a valve such as a mechanical valve or a micromechanical valve, etc. separating the compartment and the reaction site.

In some cases, additional compounds may be combined with the precursor(s) to, for example, preserve the precursor(s) against decomposition or degradation, to enhance the ability of the precursor(s) to react (e.g., a catalyst or an enzyme), or to enhance the absorption of incident energy onto the precursor(s), for instance, to increase the reaction rate of the precursor(s) to form an agent. In some embodiments, a material that is able to absorb of incident electromagnetic radiation, such as a darkened or “black” material, may be added to the precursor(s), for example, to enhance the absorption of energy. Non-limiting examples of such materials include quartz, black glass, silicon, black sand, carbon black, and the like. The additional compounds may be substantially unreactive, unable to form a transportable agent (i.e., transportable through a layer or a component of the chip), or the additional compounds may not significantly interfere with the production of the agent or with control of an environmental factor associated with the reaction site.

In some embodiments, the agent so produced may be a gas, for example, O₂, CO, CO₂, NO, NO₂, HCl, or the like. In some cases, the agent-producing reaction may produce one or more gases and/or one or more non-gaseous products. In some cases, the gaseous agent(s) may then be transported into or proximate the reaction site (for example, through a membrane or over a barrier), while non-gaseous products (such as liquids or solids) may be prevented from entering the reaction site in some fashion.

Chips of the invention may be connected to one or more fluid pathways for delivery of species or removal of species from a reaction site. In some cases, a fluidic pathway can be created in situ (after construction of the chip, during chip setup and/or during use of the chip) by permeabilizing or damaging a component separating the compartment from the reaction site (e.g., as in a wall or a membrane), or separates the compartment from a fluidic pathway in fluid communication with the reaction site. For instance, in certain embodiments of the invention, the fluidic pathway or other means for fluidic communication may be created by permeabilizing and/or damaging (reversibly or irreversibly) a component that separates the compartment containing the agent (and/or agent precursor(s)) from fluidic communication with the reaction site, or separates the compartment from a channel or other fluidic pathway in fluid communication with the reaction site, thus creating a fluidic connection between the compartment and the reaction site. For example, the component may be permeabilized by heating the component to increase the permeability of the chemical agent or by causing the component to melt or vaporize. In some cases, the permeability of the component may be enhanced by one, two, or three or more orders of magnitude. In certain cases, the permeabilization of the component may be reversible or at least partially reversible, for example, by decreasing the temperature, or introducing a non-permeabilizing agent.

The component, in some cases, may also be damaged or otherwise altered or permeabilized through a reaction, for example, a chemical or electrochemical reaction, to produce a fluidic connection with the reaction site. For example, the component may include a metal, such as gold, silver or copper, that can be electrolyzed upon the application of a suitable electrical current. As yet another example, the component may be chemically etched, for example, with a reactive species.

In still other embodiments, the component may be mechanically damaged, for example, by piercing the surface with a microneedle, which may originate from within the chip, or externally. The microneedle or other mechanical device may originate from within the chip, or externally. In one embodiment, the component may be altered on a reversible basis, for example, the component may be self-sealing and/or comprised an elastomeric substance that can be resealed.

The component may also be damaged without the use of mechanical forces or chemicals in some cases. For example, energy may be applied to the surface to damage it. In one embodiment, the component may be ablated, for example, using light. If light is used, the light may be channeled through a waveguide to the surface in some cases, or light may be applied directly to the surface. In some cases, the permeability of the component may be enhanced by one, two, or three or more orders of magnitude. In certain cases, the enhancement may be reversible, for example, by decreasing temperature, or introducing a non-permeabilizing agent.

The component may include a material able to enhance the creation of the fluidic pathway in some embodiments of the invention. As examples, the enhancing material may facilitate the absorption of light or other forms of energy, or increase the chemical reaction or transport rate. For instance, in one embodiment, the component may comprise a material that is able to absorb incident electromagnetic radiation, i.e., a darkened or “black” material, such as quartz, black glass, silicon, black sand, carbon black, and the like. As other examples, the component may include a catalyst, an enzyme, or a permeation enhancer.

In some embodiments, any of the above-described chips may be constructed and arranged such that the chip is able to respond to a change in an environmental condition within or associated with a reaction site, for example, by use of a control system. Detection of the environmental condition may occur, for example, by means of a sensor which may be positioned within the reaction site, or positioned proximate the reaction site, i.e., positioned such that the sensor is in communication with the reaction site in some manner (for example, fluidly, optically, thermally, pneumatically, electronically etc.) to the extent that it can sense one or more conditions that it is designed to sense within or associated with the reaction site. The sensor may be, for example, a pH sensor, an oxygen sensor, a sensor able to detect the concentration of a substance, or the like. The sensor may be embedded and integrally connected with the chip (e.g., within a component defining at least a portion of the reaction site a channel in fluidic communication with the reaction site, etc., or separate from the chip. In certain embodiments, the sensor may be a ratiometric sensor, i.e., a sensor able to determine a difference or ratio between two (or more) measurements.

As used herein, a “control system” is a system able to detect and/or measure one or more environmental factors within or associated with the reaction site, and cause a response or a change in the environmental conditions within or associated with the reaction site (for instance, to maintain an environmental condition at a certain value). The response produced by the control system may be based on the environmental factor in certain cases. An “active” control system, as used herein, is a system able to cause a change in an environmental factor associated with a reaction site as a direct response to a measurement of the environmental condition. The active control system may provide an agent that can be delivered, or released from the reaction, where the agent is controlled in response to a sensor able to determine a condition associated with the reaction site. A “passive” control system, as used herein, is a system able to maintain or cause a change in an environmental condition of the reaction site without requiring a measurement of an environmental factor. The passive control system may control the environmental factor within the reaction site, but not necessarily in response to a sensor or a measurement. The passive control system may allow an agent to enter or exit the reaction site without active control. For example, a passive control system may include an oxygen membrane and/or a water-permeable membrane, where the membrane can maintain the oxygen and/or the water vapor content within the reaction site, for instance, within certain predetermined limits. The control system may be able to control one or more conditions within or associated with the reaction site for any length of time, for example, 1 day, 1 week, 30 days, 60 days, 90 days, 1 year, or indefinitely in some cases.

In one aspect, the present invention provides any of the above-mentioned chips packaged in kits, optionally including instructions for use of the chips. That is, the kit can include a description of use of the chip, for example, for use with a microplate, or an apparatus adapted to handle microplates. As used herein, “instructions” can define a component of instruction and/or promotion, and typically involve written instructions on or associated with packaging of the invention. Instructions also can include any oral or electronic instructions provided in any manner such that a user of the chip will clearly recognize that the instructions are to be associated with the chip. Additionally, the kit may include other components depending on the specific application, for example, containers, adapters, syringes, needles, replacement parts, etc. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus, comprising:
a chip constructed and arranged to be rotatable about an axis passing through the chip, the chip comprising a predetermined reaction site having a volume of less than about 1 ml, wherein the predetermined reaction site is constructed and arranged to maintain at least one living cell at the site.

2. The apparatus of claim 1, wherein the chip is substantially planar.

3. The apparatus of claim 2, wherein the chip is substantially circular.

4-5. (canceled)

6. The apparatus of claim 1, wherein the predetermined reaction site has a volume of less than about 500 microliters 1 milliliter.

7. The apparatus of claim 1, wherein the predetermined reaction site has a volume of less than about 100 microliters.

8. (canceled)

9. The apparatus of claim 1, wherein the predetermined reaction site has a volume of less than about 1 microliter.

10. The apparatus of claim 1, wherein the predetermined reaction site has a maximum dimension of less than about 1 cm.

11-13. (canceled)

14. The apparatus of claim 1, wherein the predetermined reaction site comprises a cytophilic region.

15. The apparatus of claim 1, wherein the chip has a port comprising an elastomeric material in fluid communication with the predetermined reaction site.

16. The apparatus of claim 1, wherein the chip has at least two layers.

17-65. (canceled)

66. A method, comprising:
applying a differential force on a chip comprising a predetermined reaction site having a volume of less than about 1 ml, the predetermined reaction site constructed and arranged to maintain at least one living cell at the site, wherein the chip is constructed and arranged to be rotatable about an axis passing through the chip.

67-69. (canceled)

70. An apparatus, comprising:
a chip comprising a predetermined reaction site having a volume of less than 1 ml, an inlet for adding at least one living cell to the predetermined reaction site, and an outlet for release of a product of a reaction involving the at least one living cell, wherein the chip is constructed and arranged to be rotatable about an axis passing through the chip.

71-83. (canceled)

84. An apparatus as in claim 70, further comprising a mixing unit fluidly connectable to the inlet of the chamber, wherein the mixing unit is attachable to and separable from the reaction unit.

85-89. (canceled)

90. An apparatus as in claim 70, further comprising a heating and dispersion unit having an inlet, and an outlet connectable to the inlet of the chip, the heating and dispersion unit separable from and attachable to the chip.

91-93. (canceled)

94. An apparatus as in claim 70, wherein the chip has a surface adapted for immobilization of at least one cell.

95-99. (canceled)

100. An apparatus as in claim 70, further comprising at least one sensor of temperature, pH, oxygen concentration, or pressure.

101. An apparatus as in claim 100, comprising sensors of each of temperature, pH, and oxygen concentration.

102. (canceled)

103. An apparatus as in claim 100, comprising at least 10 predetermined reaction sites constructed and arranged to operate in parallel, each of the predetermined reaction sites, attachable to and separable from each other, constructed and arranged to operate in parallel.

104. An apparatus as in claim 102, comprising at least 100 predetermined reaction sites constructed and arranged to operate in parallel.

105-108. (canceled)

109. An apparatus as in claim 70, wherein the reaction comprises producing a protein.

110-113. (canceled)
